(19) United States
(12) Patent Application Publication
(10) Pub. No.: US 2016/0309871 A1
(43) Pub. Date: Oct. 27, 2016

(54) METHOD OF ACHIEVING TARGETED DELIVERY/APPLICATION OF HAIR

(71) Applicant: The Procter & Gamble Company, Cincinnati, OH (US)

(72) Inventors: Jazmin Veronica Torres Rivera, Liberty Township, OH (US); Jean Jianqun Zhao, Cincinnati, OH (US); Robert Wayne Glenn, JR., Liberty Township, OH (US)

(21) Appl. No.: 15/136,032

(22) Filed: Apr. 22, 2016

Related U.S. Application Data
(60) Provisional application No. 62/151,718, filed on Apr. 23, 2015.

(51) Int. Cl. A45D 19/02; A61Q 5/12; A61Q 5/02; A61K 8/04

(52) U.S. Cl. CPC A45D 19/02; A61K 8/046; A61Q 5/12; A61Q 5/02; A61K 2800/884; A45D 2019/0033

(57) ABSTRACT

A method of washing and conditioning hair comprising directed to applying and massaging a shampoo composition directly onto the scalp and root area of the hair, where root area is defined as 50% of the total length of the hair starting at the end attached to the scalp; applying a rinse-off conditioner composition on the length and ends of the hair, where length is defined as the hair starting below the root line at the back of the neck/base of the head and the ends are defined as the 4 cm of the hair furthest away from the scalp; and rinsing the hair with water.

### FIELD OF THE INVENTION

A method of achieving targeted delivery of a composition to the hair.

### BACKGROUND OF THE INVENTION

The condition of hair is not uniform along the entire length, particularly for consumers with previously colored or chemically treated hair. Hair strand condition varies from new growth/virgin hair at the root to older/damaged hair at the ends. Damage may be the result of mechanical (friction), environmental (sun/UV) and/or chemical (color, perms, relaxers, etc) and it is typically worse at the ends. The intermediate hair length has characteristics between these two extreme conditions.

This non-uniformity of the condition of the surface leads to other non-uniform conditions. For example, sebum load is highest at the root given its proximity to the scalp where sebum is produced. One of the reasons sebum accumulates at the root is its high affinity to the hydrophobic hair root surface. In addition, conditioning agents typically found in shampoos and conditioners are more likely to deposit at the root also due to their high affinity to hydrophobic surfaces.

Thus, there exists a need to treat hair roots differently than hair tips when washing and conditioning. Some available shampoos and conditioners suggest application to focus on scalp and ends respectively. However, Habits and Practices data shows most consumers apply shampoos and conditioners uniformly across the hair surface.

In one study, 306 consumers are asked how they apply shampoo when washing their hair. Data shows most consumers apply shampoo uniformly across hair, including scalp, roots, length and ends. In fact, only 5% of consumers apply shampoo only to scalp and roots as described in the invention.

How US Consumers apply Shampoo (n = 306)

| | |
|---|---|
| All over hair (scalp, roots, length, ends) | 78% |
| All over hair but avoiding length and ends | 16% |
| Just on scalp and roots | 5% |

In another Habits and Practices study 842 conditioner users are asked how they apply conditioner throughout the hair. Data also shows most consumers apply conditioner all over the hair, including scalp and roots.

How Conditioner Users Apply Conditioner (n = 842)

| | |
|---|---|
| All over hair, including scalp and roots | 79% |
| All over hair but avoiding roots and scalp | 12% |
| Just length and ends | 9% |

Such uniform application can lead to sub-optimal results for consumers. For example, the ends, where sebum load is lower, become over-washed or stripped from their natural lipids; b) the scalp and roots, where sebum load is higher, can be under-washed; leading to buildup and weigh down over time; c) the scalp and roots, where hair is more hydrophobic, can be over-conditioned; also leading to buildup and weigh down over time; d) the ends, where hair is less hydrophobic, can be under-conditioned; leading to hair feeling dry and brittle.

In a study, 55 consumers use a set of products using their regular application method (Control System below) and the same set of products using the targeted application method described in the invention (Targeted Application System below). Order of use is randomized to balance the order bias.

% Experienced Benefit (A/B = 90% Confidence,
a/b = 85% Confidence)

| | Targeted Appli- | Control |
|---|---|---|
| | cation System | System |
| | A | B |
| Base | 55 | 55 |
| Deep cleansing | 38 B | 24 |
| Removed build up and residue | 35 B | 16 |
| Reduced scalp itch | 15 b | 7 |
| Moisturized/Hydrated hair | 40 b | 29 |
| Reduced Split Ends | 25 B | 7 |
| Repaired Hair Damage | 25 B | 13 |
| Reduced scalp dryness | 18 B | 9 |

This data demonstrates more consumers notice benefits with the targeted application system than with the standard application method.

Thus there is a need for a method and instructions that enable non uniform application of cleansing and conditioning products throughout the hair surface.

### SUMMARY OF THE INVENTION

In an embodiment of the present invention, it is directed to a method of washing and conditioning hair comprising applying and massaging a shampoo composition directly onto the scalp and root area of the hair, where root area is defined as 50% of the total length of the hair starting at the end attached to the scalp; applying a rinse-off conditioner composition on the length and ends of the hair, where length is defined as the hair starting below the root line at the back of the neck/base of the head and the ends are defined as the 4 cm of the hair furthest away from the scalp; and rinsing the hair with water.

### DETAILED DESCRIPTION OF THE INVENTION

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography “QS” means sufficient quantity for 100%.

All numerical amounts are understood to be modified by the word “about” unless otherwise specifically indicated. Unless otherwise indicated, all measurements are understood to be made at 25° C. and at ambient conditions, where “ambient conditions” means conditions under about one atmosphere of pressure and at about 50% relative humidity. All such weights as they pertain to listed ingredients are based on the active level and do not include carriers or by-products that may be included in commercially available materials, unless otherwise specified.

Herein, “comprising” means that other steps and other ingredients which do not affect the end result can be added. This term encompasses the terms “consisting of” and “consisting essentially of”. The compositions, methods, uses, kits, and processes of the present invention can comprise, consist of, and consist essentially of the elements and limitations of the invention described herein, as well as any of the additional or optional ingredients, components, steps, or limitations described herein.

The term “substantially free from” or “substantially free of” as used herein means less than about 1%, or less than about 0.8%, or less than about 0.5%, or less than about 0.3%, or about 0%, by total weight of the composition.

Where amount ranges are given, these are to be understood as being the total amount of said ingredient in the composition, or where more than one species fall within the scope of the ingredient definition, the total amount of all ingredients fitting that definition, in the composition.

For example, if the composition comprises from 1% to 5% fatty alcohol, then a composition comprising 2% stearyl alcohol and 1% cetyl alcohol and no other fatty alcohol, would fall within this scope.

The amount of each particular ingredient or mixtures thereof described hereinafter can account for up to 100% (or 100%) of the total amount of the ingredient(s) in the hair care composition.

“Hair,” as used herein, means mammalian hair including scalp hair, facial hair and body hair, particularly on hair on the human head and scalp.

“Cosmetically acceptable,” as used herein, means that the compositions, formulations or components described are suitable for use in contact with human keratinous tissue without undue toxicity, incompatibility, instability, allergic response, and the like. All compositions described herein which have the purpose of being directly applied to keratinous tissue are limited to those being cosmetically acceptable.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. A polymer comprises at least two monomers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

“Kit,” as used herein, means a packaging unit comprising a plurality of components. An example of a kit is, for example, a first composition and a separately packaged second composition. Another kit may comprise a first composition and an energy delivery device. A different kit may comprise three different types of separately packaged composition and a hair styling implement. A further kit may comprise application instructions comprising a method and a composition/formulation.

The term “coacervate” as used herein, means the complex which forms between surfactant and polymer that may either be soluble or insoluble in the neat composition, typically forming an insoluble complex in the neat composition, and which may become less soluble upon dilution and thus yielding an increase in its level of phase separation or precipitate in solution.

The term “charge density” as used herein, means the ratio of the number of positive charges on a monomeric unit (of which a polymer is comprised) to the M.Wt. of said monomeric unit. The charge density multiplied by the polymer M.Wt. determines the number of positively charged sites on a given polymer chain. For cationic guars, charge density is measured using standard elemental analysis of percentage nitrogen known to one skilled in the art. This value of percentage nitrogen, corrected for total protein analysis, can then be used to calculate the number or equivalence of positive charges per gram of polymer. For the cationic copolymers, the charge density is a function of the monomers used in the synthesis. Standard NMR techniques know to one skilled in the art would be used to confirm that ratio of cationic and non-ionic monomers in the polymer. This would then be used to calculate the number or equivalence of positive charger per gram of polymer. Once these values are known, the charge density is reported in milliequivalence (meq) per gram of cationic polymer.

### Applicator

In the hair washing process, cleaning needs are highest at the scalp and root. Therefore, it is most beneficial to maximize the concentration of cleaning agents directly in this area. However, shampoos are applied to the outside surface of the hair and mixed with water to reduce its viscosity and enable it to travel to the scalp. Dispensing directly onto the scalp via a targeted delivery applicator enables deposition of the nondiluted cleaning agents directly where the cleaning needs are highest. This also minimizes the risk of eye contact with the cleansing solution.

The applicator is attached or can be attached to a bottle containing the cleansing product. The applicator can consist of a base that holds or extends to a single or plurality of tines. The tines have openings that may be at the tip, the base or at any point between the tip and the base. These openings allows for the product to be distributed from the bottle directly onto the hair and/or scalp.

Alternatively, the applicator can also consist of brush-like bristles attached or extending from a base. In this case product would dispense from the base and the bristles would allow for product distribution via the combing or brushing motion.

Applicators as described above may also be leveraged to enable targeted application of the conditioning product. In this case it may be most beneficial for the openings to be located at the base or between the base and the tips.

Applicator and tine design and materials can also be optimized to enable scalp massage. In this case it would be beneficial for the tine or bristles geometry at the tips to be more rounded similar to the roller ball applicator used for eye creams. It may also be beneficial for materials to be smoother and softer; for example metal or metal-like finishes, “rubbery materials”.

In an embodiment of the present invention, the root area may be a) 50% of the total length of the hair starting at the end attached to the scalp, In a further embodiment, the root area may be b) the 25 cm closest to the scalp starting from the scalp, in a further embodiment, c) the 15 cm closest to the scalp starting from the scalp, in a further embodiment d) the 5 cm closest to the scalp starting from the scalp, or in a further embodiment wherein the 25, or in an embodiment wherein b, c and d in the crown area where crown area is defined as any hair above the ear line.

### Viscosities

a. Cleansing Product—In the present invention, for the cleansing product, the viscosity may be thin enough that it easily dispenses from the package and distributes through the scalp and thick enough to stay at the root without dripping from hair as well as hands. One means to achieve excellent deep and uniform cleansing on in such a way that rheological parameters such as viscosity are low. However, such compositions would not stay where applied and would drip, run, or transfer to other areas in a manner such as to reduce its effectiveness. Thus, it has heretofore been believed that an adequate viscosity of a cleansing composition is necessary to retain cleansing composition in the defined locations as to deliver benefits.
   i. Liquid composition dispensing using hands without an applicator: The composition has a zero shear viscosity value measured at 0.01 Pa of from about 1,000 Pa s to about 20,000 Pa s, in an embodiment greater from about 1,500 Pa s to about 15,000 Pa s, in a further embodiment from about 2,000 Pa s to about 10,000 Pa s.
      In a further embodiment the composition has a zero shear viscosity measured at 0.01 Pa of less than about 20,000 Pa s; in a further embodiment, less than about 15,000 Pa s; in yet a further embodiment less than about 10,000 Pa s.
         Thin enough to cover and spread on scalp, thick enough to not drip from hands when apply.
   ii. Liquid composition dispensing with an applicator (non-foaming) The composition is shear thinning and has (1) a zero shear viscosity value measured at 0.01 Pa of from about 1,500 Pa s to about 10,000 Pa s, in an embodiment from about 2,000 Pa s to about 8,000 Pa s, and in a further embodiment from about 2,500 Pa s to about 6,000 Pa s; (2) a moderate stress viscosity value measured at 1 Pa viscosity of greater than about 500 Pa s and less than 5,000 Ps, in an embodiment from about 600 Pa s to about 4,500 Pa s, in a further embodiment from about 700 Pa s to about 4,000 Pa s; and (3) a high shear rate viscosity value measured at 100 1/s of less than about 100 Pa s, in an embodiment less than about 70 Pa s, and in a further embodiment less than about 50 Pa s.
      Shear thinning through the applicator for easy of dispensing
Foam enables low viscosity uniform spreading, lathering and coverage on scalp and through the hair, not dripping and wherein viscosity is low enough for foaming.
   iii. Liquid composition dispensing with a foam applicator The composition has a zero shear viscosity value measured at 0.01 Pa of less than about 15,000 Pa s, in an embodiment less than about 10,000 Pa s, in a further embodiment less than about 8,000 Pa S.
b. Conditioning Product—In the present invention, the viscosity of a conditioning product may be thin enough that it easily dispenses from the package while thick enough that it does not drip from the hair.
   One means to achieve excellent rich and uniform conditioning of the hair ends/tips on in such a way that rheological parameters such as viscosity are preferred to be high. However, such compositions would not be able to dispense from package, esp. applicators and would not uniformly spread through the hair where the hair can be heavy and weigh down. Thus, it has heretofore been believed that an adequate viscosity of a conditioning composition is necessary to achieve uniform richness of conditioning as to deliver benefits.
   i. Liquid composition dispensing using hands without an applicator: The composition may have a zero shear viscosity value measured at 0.01 Pa of from about 4,500 Pa s to about 50,000 Pa s, from about 5,000 Pa s to about 30,000 Pa s, and in a further embodiment from about 6,000 Pa s to about 20,000 Pa s.
      Such a liquid composition may be thick enough to not drip from hair.
   ii. Liquid composition dispensing with an applicator (non-foaming) The composition may be shear thinning and has (1) a zero shear viscosity value measured at 0.01 Pa of greater than about 3,500 Pa s to about 50,000 Pa s, in an embodiment from about 4,000 Pa s to about 30,000 Pa s, and in a further embodiment from about 5,000 Pa s to about 20,000 Pa s; (2) a moderate stress viscosity value measured at 1 Pa viscosity of greater than about 1,000 Pa s and less than 15,000 Ps, in an embodiment from about 1,500 Pa s to about 13,500 Pa s, in a further embodiment from about 2,000 Pa s to about 12,000 Pa s; and (3) a high shear rate viscosity value measured at 100 l/s of less than about 800 Pa s, in an embodiment less than about 700 Pa s, in a further embodiment less than about 600 Pa s.

Such a composition may provide shear thinning through the applicator for ease of dispensing. Likewise, such a composition may be thick enough for not dripping from hair.

Foam may provide low viscosity uniform spreading, lathering and coverage on scalp and through the hair, not dripping. Viscosity needs to be low enough for foaming
iii. Liquid composition dispensing with a foam applicator

Such a composition may have a zero shear viscosity value measured at 0.01 Pa of less than 15,000 Pa s, in an embodiment less than about 10,000 Pa s, in a further embodiment less than about 8,000 Pa s.

### For Compositions

a. Cleansing Product for Scalp and Root Application—may provide high cleaning/low conditioning. Such compositions may comprise a low to high weight percentage of hair conditioning agents. The hair conditioning agents may include, but not limited, to include silicones, cationic polymers and scalp health actives. Non-limiting examples are provided in the tables below which demonstrate:
   i. Examples of cleansing liquid composition for dispensing using hands without an applicator;
   ii. Examples of cleansing liquid composition for dispensing with an applicator (non-foaming);
   iii. Examples of cleansing liquid composition for dispensing with a foam applicator.
b. Conditioning Product for Tips/Ends Application—may provide high conditioning. Such compositions may comprise a low to high weight percentage of hair conditioning agents. The hair conditioning agents may include, but not limited, to include silicones, cationic polymers and scalp health actives. Non-limiting examples are provided in the tables below which demonstrate:
   i. Examples of conditioning liquid composition for dispensing using hands without an applicator:
   ii. Examples of conditioning liquid composition for dispensing with an applicator (non-foaming);
   iii. Examples of conditioning liquid composition for dispensing with a foam applicator.

### Dilution of Cleansing Product

Non uniform cleaning can also be achieved by diluting the first cleansing product and applying it to the ends. This can be achieved by mixing the first cleansing product with a diluting component comprising water and optionally at least one active component. The dilution may occur in a separate container or in a built in compartment in the primary package.

This regimen of non homogenous application may be presented as 2 products in a dual chamber package. Dual chamber packs have an outer design that appears and can be used as a standard bottle but contain a typically inner physical barrier that enables it to contain two products at the same time and keep them separated. These may include a dial to switch from one product to the other or to mix the two products together at different ratios.

### EXAMPLES

The following examples illustrate the present invention. The exemplified compositions can be prepared by conventional formulation and mixing techniques. It will be appreciated that other modifications of the present invention within the skill of those in the hair care formulation art can be undertaken without departing from the spirit and scope of this invention. All parts, percentages, and ratios herein are by weight unless otherwise specified. Some components may come from suppliers as dilute solutions. The levels given reflect the weight percent of the active material, unless otherwise specified.

### NON-LIMITING EXAMPLES

### EXAMPLES

### Rinse Off Cleansing Shampoo Examples

| Ingredient | Ex. 1 | Ex. 2 | Ex. 3 | Ex. 4 | Ex. 5 | Ex. 6 | Ex. 7 | Ex. 8 | Ex. 9 | Ex. 10 |
|---|---|---|---|---|---|---|---|---|---|---|
| Water | q.s. | q.s. | q.s. | q.s. | q.s. | q.s. | q.s. | q.s. | q.s. | q.s. |
| Sodium Laureth Sulfate | 7.9 | 6 | 10 | 10 | — | — | 6 | — | — | 10 |
| (SLE3S-28% active)¹ | | | | | | | | | | |
| Sodium Laureth Sulfate | — | — | — | — | 13 | 11 | — | 12 | 10.5 | — |
| (SLE1S-29% active) ² | | | | | | | | | | |
| Sodium Lauryl Sulfate | 5.7 | 3.5 | 7 | 7 | — | — | 7 | — | 1.5 | 1.5 |
| (SLS-29% active) ³ | | | | | | | | | | |
| Coco monoethanolamide | 0.5 | 0.85 | 0.4 | 0.4 | — | — | — | 1 | 0.8 | — |
| (85% active)⁴ | | | | | | | | | | |
| Cocoamdopropyl Betaine | 2.1 | 0.75 | — | — | 1 | 1.5 | 1 | 1 | 2 | 2 |
| (30% active)⁵ | | | | | | | | | | |
| Ethylene Glycol Disterate⁶ | — | — | 1.5 | 1.5 | — | — | 1.5 | 1.5 | 1.5 | 1.5 |
| Polyquaternium 76⁷ | — | 0.25 | — | — | — | — | 0.01 | — | — | — |
| Polquaternium 10⁸ | — | — | 0.25 | 0.25 | — | — | — | — | — | — |
| Polyquaternium 6⁹ | — | — | — | — | 0.1 | — | — | — | — | — |
| Guar, Hydroxylpropyl | — | — | — | — | — | 0.2 | — | 0.4 | — | 0.15 |
| Trimonium Chloride¹⁰ | | | | | | | | | | |
| Guar, Hydroxylpropyl | — | — | — | — | — | — | — | — | 0.25 | — |
| Trimonium Chloride¹¹ | | | | | | | | | | |
| 330M silicone 712¹² | — | 1.5 | — | 1.5 | — | — | 0.75 | — | 1 | 1 |
| Silicone microemulsion | — | — | — | — | — | 1.4 | — | — | — | — |
| (35% active)¹³ | | | | | | | | | | |
| Trihydroxysteam¹⁴ | — | — | 0.25 | — | 0.25 | 0.25 | — | — | — | — |
| Zinc Pyrithone¹⁵ | — | — | — | — | — | — | 1 | 1 | 1 | — |
| Zinc Carbonate¹⁶ | — | — | — | — | — | — | 1.6 | 1.6 | 1.6 | — |
| Gel Network¹⁷ | — | — | — | — | — | — | — | — | 27.3 | 20.2 |
| Menthol¹⁸ | — | — | — | — | — | — | 0.3 | — | — | — |
| Fragrance | 0.7 | 0.7 | 0.6 | 0.7 | 0.7 | 0.7 | 0.7 | 0.7 | 0.7 | 0.7 |

| | |
|---|---|
| Sodium Chloride¹⁹ | Adjust as needed for viscosity |
| Preservatives, pH adjusters | Up to 1% |

¹Sodium Laureth (3 molar ethylene oxide) Sulfate at 28% active, supplier: P&G
² Sodium Laureth (1 molar ethylene oxide) sulfate at 29% active, supplier: P&G
³ Sodium Lauryl Sulfate at 29% active, supplier: P&G
⁴Coco monethanolamide at 85% active, supplier: Stephan Co or supplier Evonik
⁵Tegobetaine F-B, 30% active, supplier: Goldschmidt Chemical
⁶Ethylene Glycol Disterate at 100% active, supplier: Goldschmidt Chemical or supplier Evonik
⁷Acrylamide: Triquat cationic polymer, trade name: Mirapol AT from Rhodia,
⁸KG30M cationic cellulose polymer from Amerchol Dow
⁹Polydadmac, trade name: Mirapol 1005 from Rhodia
¹⁰Jaguar C500 from Rhodia
¹¹Jaguar C17 from Rhodia
¹²330M silicone, 100% active, supplier: Momentive (silicone used by P&G to make a 70% active, 30 um emulsion)
¹³Belsil 3560 VP silicone microemulsion (35% active) from Wacker, 60,000 cst internal viscosity of silicone, particle size approx. 125 nm
¹⁴Thixin R from Rheox Inc.
¹⁵ZPT from Arch Chemical
¹⁶Zinc carbonate from the Bruggeman Group
¹⁷Gel Networks; See Composition below. The water is heated to about 74° C. and the Cetyl Alcohol, Stearyl Alcohol, and the SLES Surfactant are added to it. After incorporation, this mixture is passed through a heat exchanger where it is cooled to about 35° C. As a result of this cooling step, the Fatty Alcohols and surfactant crystallized to form a crystalline gel network.
¹⁸Menthol from symrise
¹⁹Sodium Chloride USP (food grade) from Morton

| Ingredient | Wt % |
|---|---|
| Water | q.s. |
| Cetyl Alcohol | 3.46 |
| Steary Alcohol | 6.44 |
| Sodium Laureth Sulfate | 3.93 |
| (SLE3S-28% active) | |
| Preservatives | 0.03 |

### Rinse Off Aerosol Foam Shampoo Examples

| Ingredient | Ex. 21 | Ex. 22 | Ex. 23 | Ex. 24 | Ex. 25 |
|---|---|---|---|---|---|
| Water | q.s. | q.s. | q.s. | q.s. | q.s. |
| Sodium Undecyl Sulfate | 24 | 24 | 24 | 24 | — |
| (C11 70% active)¹ | | | | | |
| Sodium Laureth Sulfate | — | — | — | — | 24 |
| (SLE1S-70% active) ² | | | | | |
| Lauramidopropyl Betaine | 6 | 6 | 6 | 6 | — |
| (LAPB 35% active)³ | | | | | |
| Coco monoethanolamide | — | — | — | — | 2 |
| (85% active)⁴ | | | | | |
| Lauryl Hydroxysultaine | — | — | — | — | 4 |
| (42.5% active)⁵ | | | | | |
| Para Hydroxy Phenyl | 4 | 4 | 4 | 4 | 4 |
| Butanone6 | | | | | |
| Guar, Hydroxylpropyl | — | — | 0.4 | 0.4 | |
| Trimonium Chloride⁷ | | | | | |
| Polyquatenrium 8 | — | — | — | 0.2 | — |
| Silicone quaternium 9 | — | — | — | 2 | — |
| Fragrance | 2.4 | 2.4 | 2.4 | 2.4 | 2.4 |
| Sodium Chloride¹⁰ | Adjust as needed for viscosity | | | | |
| Preservatives, | Up to 1% | | | | |
| pH adjusters | | | | | |
| Propellant A46 ¹¹ | 4 | | 4 | 4 | 4 |
| Propellant HFO ¹² | | 4 | | | |

¹Sodium Undecyl Sulfate (C11, Isachem 123S) at 70% active, supplier: P&G
² Sodium Laureth (1 molar ethylene oxide) sulfate at 70% active, supplier: Stephan Co
³LAPB (Mackam DAB), at 35% active level, supplier: Rhodia
⁴Coco monethanolamide at 85% active, supplier: Stephan Co or supplier Evonik
⁵LHS (Mackam LHS) at 42.5% active level, supplier: Rhodia
6Raspberry Ketone, supplier: Spectrum
⁷Jaguar C500, from Rhodia
8. Polydadmac, trade name: Mirapol 100s, from Rhodia
9. Silicone quaternium-22, Abil ME 45 from Evonik
¹⁰Sodium Chloride USP (food grade) from Morton
¹¹ A46 (Isobutane to Propane = 84.8 to 15.2) from Diversified CPC International
¹² Hydrofluoroolefin (HFO-1234ze) from Honeywell

### Rinse Off Conditioner Examples

| Ingredient | Ex. 11 | Ex. 12 | Ex. 13 | Ex. 14 | Ex. 15 | Ex. 16 | Ex. 17 | Ex. 18 | Ex. 19 | Ex. 20 |
|---|---|---|---|---|---|---|---|---|---|---|
| Water | q.s. | q.s. | q.s. | q.s. | q.s. | q.s. | q.s. | q.s. | q.s. | q.s. |
| Behentrimonium chloride ¹ | 2.3 | 2.8 | 3.4 | — | 1.8 | — | — | — | — | — |
| Behentrimonium methosulfate ² | — | — | — | 2.8 | — | 2.2 | — | 3.0 | — | — |
| Stearamidopropyl dimethylamine ³ | — | — | — | — | — | — | 3.24 | — | — | 1.0 |
| Behenamidopropyl dimethylamine ⁴ | — | — | — | — | — | — | — | — | 2.5 | — |
| Dicetyldimonium chloride ⁵ | — | — | — | — | — | 0.74 | — | — | — | — |
| Distearyldimonium chloride ⁶ | — | — | — | — | — | — | — | — | 0.6 | 0.7 |
| Cetyl alcohol ⁷ | 1.5 | 1.9 | 2.2 | 1.9 | 1.2 | 1.2 | 4.3 | 1.0 | 1.5 | 1.2 |
| Stearyl alcohol ⁸ | 3.7 | 4.7 | 5.5 | 4.6 | 2.9 | 2.94 | 2.9 | 2.5 | 4.0 | 0.8 |
| Dipropylene glycol ⁹ | — | — | 0.7 | — | — | — | — | — | — | — |
| Glycerin ¹⁰ | — | — | — | — | — | 3.0 | 3.0 | — | — | — |
| Butylene glycol¹¹ | — | — | — | — | — | — | — | 2.0 | — | — |
| EDTA ¹² | 0.1 | 0.1 | 0.1 | 0.1 | 0.1 | 0.1 | 0.1 | 0.1 | 0.1 | 0.1 |
| Aminosilicone ¹³ | 1.5 | 1.5 | 1.5 | 1.5 | 1.5 | 1.5 | 1.5 | 1.5 | 0.5 | 0.5 |
| L-glutamic acid ¹⁴ | — | — | — | — | — | — | 1.04 | — | 0.7 | — |
| Panthenol ¹⁵ | 0.05 | 0.05 | 0.05 | 0.05 | 0.05 | 0.05 | 0.05 | 0.05 | 0.05 | 0.05 |
| Panthenyl ethyl ether ¹⁶ | 0.03 | 0.03 | 0.03 | 0.03 | 0.03 | 0.03 | 0.03 | 0.03 | 0.03 | 0.03 |
| Fragrance | 0.5 | 0.5 | 0.5 | 0.5 | 0.5 | 0.5 | 0.5 | 0.5 | 0.5 | 0.5 |

| | |
|---|---|
| Preservatives, pH adjusters | Up to 1% |

¹ BTMAC (Genamin KDMP), available from Clariant
² BTMS (Genamin BTMS), available from Clariant
³ SAPDMA (IncromineSD), available from Croda
⁴ BAPDMA (IncromineBB), available from Croda
⁵ DCDMC (Varisoft 432), available from Evonik
⁶ DSDMC (Varisoft TA100), available from Evonik
⁷ Cetyl alcohol, available from Procter & Gamble
⁸ Stearyl alcohol, available from Procter & Gamble
⁹ Dipropylene glycol, available from Procter & Gamble
¹⁰ Glycerin, available from Procter & Gamble
¹¹Butylene glycol, available from Procter & Gamble
¹² Ethylene Diamine Tetraacetic Acid (EDTA), available from BASF
¹³ Aminosilicone: Available from GE having a viscosity 10,000 mPa · s, and having following formula (I): (R₁)ₐG₃₋ₐ—Si—(—OSiG₂)ₙ—(—OSiG_{b}(R₁)_{2-b})ₘ—O—SiG₃₋ₐ(R₁)ₐ (I) wherein G is methyl; a is an integer of 1; b is 0, 1 or 2, preferably 1; n is a number from 400 to about 600; m is an integer of 0; R₁ is a monovalent radical conforming to the general formula CqH_{2q}L, wherein q is an integer of 3 and L is —NH₂
¹⁴ L-Glutamic Acid, available from Orsan/Amylum Liquid
¹⁵ dl-Panthenol (56% active), available from Dow Benelux
¹⁶ dl-Pantyl, available from Dow Benelux

### Rinse Off Aerosol Foam Conditioner Examples

| Ingredient | Ex. 31 | Ex. 32 | Ex. 33 | Ex. 34 | Ex. 35 | Ex. 36 | Ex. 37 | Ex. 38 |
|---|---|---|---|---|---|---|---|---|
| Water | q.s. | q.s. | q.s. | q.s. | q.s. | q.s. | q.s. | q.s. |
| Cetrimonium chloride ¹ | — | 2.5 | 2.5 | 2.5 | 1.0 | — | — | 1.0 |
| Behentrimonium chloride ² | — | — | — | — | 1.5 | 2.5 | 2.5 | 2.5 |
| Distearyldimonium chloride ³ | — | — | — | — | 1.0 | — | 1.0 | — |
| Cetyl alcohol ⁴ | — | — | 1.5 | 3 | — | — | — | — |
| Stearyl alcohol ⁵ | — | — | 1.5 | 3 | — | — | — | — |
| Aminosilicone micro-emulsion ⁶ | 12 | 12 | 12 | 12 | 12 | 12 | 12 | 12 |
| Propellant ⁷ | 6 | 6 | 6 | 6 | 6 | 6 | 6 | 6 |
| Fragrance | 2.4 | 2.4 | 3 | 3 | 2.4 | 2.4 | 2.4 | 2.4 |

| | |
|---|---|
| Preservatives, pH adjusters | Up to 1% |

¹ CTAC (Varisoft 100), available from Evonik
² BTMAC (Genamin KDMP), available from Clariant
³ DSDMC (Varisoft TA100), available from Evonik
⁴ Cetyl alcohol, available from Procter & Gamble
⁵ Stearyl alcohol, available from Procter & Gamble
⁶ Aminosilicone micro-emulsion (Silsoft 253), available from Momentive
⁷ A46 (Isobutane/Propane = 84.8/15.2) available from Diversified CPC International

### Viscosity Test Method

Method Which Provides a Zero Shear Viscosity Value

A zero shear viscosity as related to sedimentation and stability can be measured using the following method:

The viscosity of the scalp care composition may be determined by a cone and plate viscometer/rheometer which measures the viscous drag resulting from the sample material contained in the gap between a rotating cone and a stationary plate. The geometry of the cone and plate may be such that the entire sample is subjected to a uniform shear rate. To determine the relevant viscosity for stability and sedimentation an Advanced Rheometer 2000 fitted with a 4 degree, 6 centimeter Acrylic cone at 25 C. temperature using a 3.95 mL sample size and a solvent trap may be used with a procedure consists of a 2 minute relaxation time, followed by a constant stress creep step at 0.01 Pa. Using Stokes law, it can be calculated that the stress from gravity (sedimentation) on a particle with a radius of 2 micrometers and density 1800 kg/m³ in a fluid of density 920 kg/m³ can be approximated as 0.01 Pa. Shear rate is then determined by fitting a straight line through the strain versus time data collected from 60 seconds through 240 seconds using the Rheology Advantage Data Analysis package and viscosity at the specified stress is then calculated by stress/rate in Pa s.

Method Which Provides a Moderate Stress Viscosity Value

A moderate stress viscosity as related to dripping of a scalp care composition can be measured using the following method:

The viscosity of the scalp care composition may be determined by a cone and plate viscometer/rheometer which measures the viscous drag resulting from the sample material contained in the gap between a rotating cone and a stationary plate. The geometry of the cone and plate may be such that the entire sample is subjected to a uniform shear rate. To determine the relevant viscosity for dripping, an Advanced Rheometer 2000 fitted with a 4 degree, 6 centimeter Acrylic cone at 25 C. temperature using a 3.95 mL sample size and a solvent trap may be used with a procedure consists of a 2 minute relaxation time, followed by application of a constant stress of 1 Pa. A stress of approximately 1 Pa can be calculate based upon the stress from gravity of a droplet of fluid of density 920 kg/m³ on an angle such as one of 45 degrees. Shear rate is then determined by fitting a straight line through the strain versus time data collected from 60 seconds through 240 seconds using the Rheology Advantage Data Analysis package and viscosity at the specified stress is then calculated by stress/rate in Pa s.

Method Which Provides a High Shear Rate Viscosity Value

A high shear rate viscosity as related to spreading of a scalp care composition can be measured using the following method:

The viscosity of the hair care composition may be determined by a concentric cylinder or cup and bob viscometer/rheometer which measures the viscous drag resulting from the sample material contained in the gap between a rotating bob and a stationary cup. To determine the relevant viscosity for spreading, an Advanced Rheometer 2000 can be fitted with an aluminum bob of diameter 28 mm and cup of diameter 30 mm is held at 25 C. temperature and a sample volume of 19.6 mL. The procedure consists of a 2 minute relaxation time, followed by a peak flow hold at a constant shear rate of 100 l/s to approximate the rate at which product may be massaged into the affected area. Viscosity is measured every 10 seconds over a 1 minute time period and the Rheology Advantage Data Analysis package is used to calculate the mean viscosity in Pa s.

### Scalp Health Actives

In an embodiment of the present invention, the present invention may further comprise one or more additional scalp health actives. This group of materials is varied and provides a wide range of benefits including moisturization, barrier improvement, vitamins, lipid soluble vitamins, anti-dandruff, anti-fungal, anti-microbial, anti-oxidant, anti-itch, and sensates. Such skin health actives include but are not limited to: vitamin E and F, salicylic acid, glycols, glycolic acid, PCA, PEGs, erythritol, glycerin, lactates, niacinamide, hyaluronates, allantoin and other ureas, betaines, sorbitol, glutamates, xylitols, menthol, menthyl lactate, iso cyclomone, benzyl alcohol, and natural extracts/oils including peppermint, spearmint, argan, jojoba, chelants, perfumes, brighteners, enzymes, sensates, attractants, dyes, pigments, bleaches, aloe, a compound comprising the following structure:

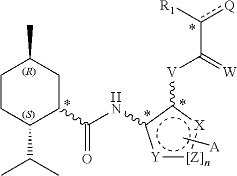

R₁ is selected from H, alkyl, amino alkyl, alkoxy;

Q=H₂, O, —OR₁, —N(R₁)₂, —OPO(OR₁)ₓ, —PO(OR₁)ₓ, —P(OR₁)ₓ where x=1-2;

V=NR₁, O, —OPO(OR₁)ₓ, —PO(OR₁)ₓ, —P(OR₁)ₓ where x=1-2;

W=H₂, O;

X, Y=independently selected from H, aryl, naphthyl for n=0;

X, Y=aliphatic CH₂ or aromatic CH for n≧1 and Z is selected from aliphatic CH₂, aromatic CH, or heteroatom;

A=lower alkoxy, lower alkylthio, aryl, subsitituted aryl or fused aryl; and

stereochemistry is variable at the positions marked*; natural extracts/oils including peppermint, spearmint, argan, jojoba, aloe. and mixtures thereof.

### Hair Care Compositions

In an embodiment of the present invention, a rinse-off hair care composition or shampoo composition may include one or more detersive surfactants. The detersive surfactant component can be included to provide cleaning performance to the product. The detersive surfactant component in turn comprises anionic detersive surfactant, zwitterionic, amphoteric detersive surfactant, nonionic or a combination thereof.

Exemplary anionic surfactants for use in the hair care composition include ammonium lauryl sulfate, ammonium laureth sulfate, ammonium C10-15 pareth sulfate, ammonium C10-15 alkyl sulfate, ammonium C11-15 alkyl sulfate, ammonium decyl sulfate, ammonium deceth sulfate, ammonium undecyl sulfate, ammonium undeceth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, sodium C10-15 pareth sulfate, sodium C10-15 alkyl sulfate, sodium C11-15 alkyl sulfate, sodium decyl sulfate, sodium deceth sulfate, sodium undecyl sulfate, sodium undeceth sulfate, potassium lauryl sulfate, potassium laureth sulfate, potassium C10-15 pareth sulfate, potassium C10-15 alkyl sulfate, potassium C11-15 alkyl sulfate, potassium decyl sulfate, potassium deceth sulfate, potassium undecyl sulfate, potassium undeceth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, sodium dodecyl benzene sulfonate, sodium cocoyl isethionate and combinations thereof. In a further embodiment, the anionic surfactant is sodium lauryl sulfate or sodium laureth sulfate.

The composition of the present invention can also include anionic surfactants selected from the group consisting of:

a) R₁O(CH₂CHR₃O)_{y} SO₃M;

b) CH₃(CH₂)_{z} CHR₂ CH₂O(CH₂ CHR₃O)_{y} SO₃M; and

c) mixtures thereof,

where R₁ represents CH₃ (CH₂)₁₀ , R₂ represents H or a hydrocarbon radical comprising 1 to 4 carbon atoms such that the sum of the carbon atoms in z and R₂ is 8, R₃ is H or CH₃, y is 0 to 7, the average value of y is about 1 when y is not zero (0), and M is a monovalent or divalent, positively-charged cation.

Amphoteric detersive surfactants suitable for use in the rinse-off hair care compositions are well known in the art, and include those surfactants broadly described as derivatives of aliphatic secondary and tertiary amines in which an aliphatic radical can be straight or branched chain and wherein an aliphatic substituent can contain from about 8 to about 18 carbon atoms such that one carbon atom can contain an anionic water solubilizing group, e.g., carboxy, sulfonate, sulfate, phosphate, or phosphonate. Examples of compounds falling within this definition can be sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and products described in U.S. Pat. No. 2,528,378. Other examples of amphoteric surfactants can include sodium lauroamphoacetate, sodium cocoamphoactetate, disodium lauroamphoacetate disodium cocodiamphoacetate, and mixtures thereof. Amphoacetates and diamphoacetates can also be used.

Zwitterionic detersive surfactants suitable for use in the rinse-off hair care compositions are well known in the art, and include those surfactants broadly described as derivatives of aliphatic quaternary ammonium, phosphonium, and sulfonium compounds, in which aliphatic radicals can be straight or branched chains, and wherein an aliphatic substituent can contain from about 8 to about 18 carbon atoms such that one carbon atom can contain an anionic group, e.g., carboxy, sulfonate, sulfate, phosphate, or phosphonate. Other zwitterionic surfactants can include betaines, including non-limiting examples such as cocoamidopropyl betaine, lauroamidopropyl betaine.

### Rinse-Off Conditioner Composition

In an embodiment of the present invention, a rinse-off conditioner composition may also comprise a conditioner gel matrix comprising part or all of the cationic surfactant, whereas the conditioner gel network may also comprise one or more high melting point fatty compounds (i.e. fatty alcohols), and an second aqueous carrier.

The conditioner gel matrix of the conditioner composition includes a cationic surfactant or a cationic surfactant system. The cationic surfactant system can be selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt. The cationic surfactant system may be included in the composition at a level by weight of from about 0.1% to about 10%, from about 0.5% to about 8%, from about 0.8% to about 5%, and from about 1.0% to about 4%.

The conditioner gel matrix of the rinse-off conditioner composition may include one or more high melting point fatty compounds. Suitable fatty alcohols include, for example, cetyl alcohol, stearyl alcohol, behenyl alcohol, and mixtures thereof. The high melting point fatty compound can be included in the conditioner composition at a level of from about 0.1% to about 20%, alternatively from about 1% to about 15%, and alternatively from about 1.5% to about 8% by weight of the composition. The conditioner gel matrix of the conditioner composition includes a second aqueous carrier. The second aqueous carrier may comprise water, or a miscible mixture of water and organic solvent.

### Leave-On Conditioner Composition

In an embodiment of the present invention, a leave-on-conditioner composition may comprise a cationic surfactant or a mixture of cationic surfactants and an aqueous carrier. The leave-on treatment may also comprise one or more rheology modifiers and a third aqueous carrier.

In one embodiment the leave-on treatment may include a conditioner gel matrix as described above (in the rinse-off conditioner description). In another embodiment the leave-on treatment may include one or more rheology modifiers. Any suitable rheology modifier can be used. In an embodiment, the leave-on treatment may comprise from about 0.01% to about 3% of a rheology modifier, alternatively from about 0.1% to about 1% of a rheology modifier,

A. Optional Ingredients

In accordance with embodiments of the present invention, the hair care composition may further comprise one or more optional ingredients, including benefit agents Suitable benefit agents include, but are not limited to conditioning agents (silicone or non-silicone conditioning agents), cationic polymers, non-limiting examples including natural cationic deposition polymers, synthetic cationic deposition polymer, silicone emulsions, gel networks, chelating agents, and , natural oils such as sun flower oil or castor oil. Additional suitable optional ingredients include but are not limited to perfumes, perfume microcapsules, colorants, particles, anti-microbials, foam busters, anti-static agents, rheology modifiers and thickeners, suspension materials and structurants, pH adjusting agents and buffers, preservatives, pearlescent agents, solvents, diluents, anti-oxidants, vitamins and combinations thereof.

Such optional ingredients should be physically and chemically compatible with the components of the composition, and should not otherwise unduly impair product stability, aesthetics, or performance The CTFA Cosmetic Ingredient Handbook, Tenth Edition (published by the Cosmetic, Toiletry, and Fragrance Association, Inc., Washington, D.C.) (2004) (hereinafter “CTFA”), describes a wide variety of nonlimiting materials that can be added to the composition herein.

In one aspect said benefit agent may comprise an anti-dandruff agent. Such anti-dandruff particulate should be physically and chemically compatible with the components of the composition, and should not otherwise unduly impair product stability, aesthetics or performance.

According to an embodiment, the hair care composition comprises an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt.

Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt. % to about 5 wt. %, or from about 0.1 wt. % to about 3 wt. %, or from about 0.1 wt. % to about 2 wt. %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, fcharcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active is included in an amount of from about 0.01 wt. % to about 5 wt. %, or from about 0.1 wt. % to about 3 wt. %, or from about 0.3 wt. % to about 2 wt. %. In an embodiment, the azole anti-microbial active is ketoconazole. In an embodiment, the sole anti-microbial active is ketoconazole.

Embodiments of the hair care composition may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001 wt. % to about 10 wt. %, or from about 0.01 wt. % to about 7 wt. %, or from about 0.1 wt. % to about 5 wt. % of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}.nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺Aⁿ⁻_{(1=3y)/n}. nH₂O where the two metal ions (M²⁺) may be the same or different. If they are the same and represented by zinc, the formula simplifies to [Zn₁₊ₓ(OH)₂]^{2x+}2x A⁻. nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

In an embodiment, the shampoo used in the regimen contains detersive surfactant in a concentration of 15-50%.

In another embodiment the shampoo used in the regimen is delivered in a foam form via an aerosol dispenser, a pump dispenser or a squeeze bottle. Typically, relatively low viscosities of the compositions are required for foam delivery via these dispensers compared to delivery via the traditional liquid form.

Delivery of cleansing and conditioner composition in the form of foam represents an attractive consumer concept. For cleansing compositions, the low density of the foam requires relatively high concentration of surfactant to deliver sufficient amount of detersive surfactant for each use. Analogously, for conditioner compositions delivered as foams, a relatively high concentration of the conditioning agent is required.

### Shampoo Compositions

### Non-Aerosol Shampoo Compositions

| Ingredient | Ex. 1 | Ex. 2 |
|---|---|---|
| Sodium Undecyl Sulfate¹ | 24 | 24 |
| Lauramidopropyl Betaine² | 6 | 6 |
| Para Hydroxy Phenyl | 4 | — |
| Butanone³ | | |
| Dipropylene Glycol | — | 4 |
| Guar, Hydroxylpropyl | 0.4 | 0.4 |
| Trimonium Chloride⁴ | | |
| Fragrance | 2.4 | 2.4 |
| Preservatives, | Up to 1% | Up to 1% |
| pH adjusters | | |

| | |
|---|---|
| Sodium Chloride | Add as needed to adjust viscosity |

| | | |
|---|---|---|
| Water | Q.S. | Q.S. |

¹Sodium Undecyl Sulfate (from C11, Isachem 123S); supplied by P&G.
²LAPB (Mackam DAB), at 35% active level; supplied by Rhodia.
³Raspberry Ketone; supplied by Spectrum.
⁴Jaguar C500, MW of 500,000, Charge Density of 0.8; supplied by Rhodia.

| Ingredient | Ex. 3 | Ex. 4 | Ex. 5 | Ex. 6 |
|---|---|---|---|---|
| Sodium Laureth Sulfate¹ | 24 | 24 | 20 | 16 |
| Cocoamidopropyl | 4 | 4 | — | — |
| Betaine² | | | | |
| Coco | — | — | — | 1 |
| monoethanolamide³ | | | | |
| Lauryl Hydroxysultaine⁴ | 4 | 4 | — | — |
| Sodium | — | — | 10 | 8 |
| Lauroamphoacetate⁵ | | | | |
| Para Hydroxy Phenyl | — | — | 3 | 3 |
| Butanone⁶ | | | | |
| Dipropylene Glycol | 10 | 7.5 | 5 | 5 |
| Guar, Hydroxylpropyl | 0.4 | 0.4 | 0.4 | 0.4 |
| Trimonium Chloride⁷ | | | | |
| Fragrance | 2.4 | 2.4 | 2.4 | 2.4 |
| Preservatives, | Up to 1% | Up to 1% | Up to 1% | Up to 1% |
| pH adjusters | | | | |
| Sodium Chloride | Add as needed to adjust viscosity | | | |
| Preservatives, | Up to 1% | | | |
| pH adjusters | | | | |
| Water | Q.S. | Q.S. | Q.S. | Q.S. |

¹Sodium Laureth (1 molar ethylene oxide) sulfate.
²Tegobetaine F-B, 30% active; supplied by Goldschmidt Chemical.
³Coco monethanolamide at 85% active; supplied by Stephan Co.
⁴LHS (Mackam LHS) at 42.5% active level; supplied by Rhodia.
⁵NaLaa (Mackam HPL-ULS) at 22% active level; supplied by Rhodia.
⁶Raspberry Ketone; supplied by Spectrum.
⁷Jaguar C500, MW of 500,000, Charge Density of 0.8; supplied by Rhodia.

### Aerosol Shampoo Compositions

| Ingredient | Ex. 7 | Ex. 8 |
|---|---|---|
| Sodium Undecyl Sulfate¹ | 24 | 24 |
| Lauramidopropyl Betaine² | 6 | 6 |
| Para Hydroxy Phenyl | 4 | — |
| Butanone³ | | |
| Dipropylene Glycol | — | 4 |
| Guar, Hydroxylpropyl | 0.4 | 0.4 |
| Trimonium Chloride⁴ | | |
| Fragrance | 2.4 | 2.4 |
| Propellant A46 ⁵ | 4 | 4 |
| Preservatives, | Up to 1% | Up to 1% |
| pH adjusters | | |

| | |
|---|---|
| Sodium Chloride | Add as needed to adjust viscosity |

| | | |
|---|---|---|
| Water | Q.S. | Q.S. |

¹Sodium Undecyl Sulfate (from C11, Isachem 123S); supplied by P&G.
²LAPB (Mackam DAB), at 35% active level; supplied by Rhodia.
³Raspberry Ketone; supplied by Spectrum.
⁴Jaguar C500, MW of 500,000, Charge Density of 0.8; supplied by Rhodia.
⁵ Aeron A-Blends, A46 (Isobutane/Propane = 84.85/15.15); supplied by Diversified CPC International.

| Ingredient | Ex. 9 | Ex. 10 | Ex. 11 | Ex. 12 |
|---|---|---|---|---|
| Sodium Laureth Sulfate¹ | 24 | 24 | 20 | 16 |
| Cocoamidopropyl | 4 | 4 | — | — |
| Betaine² | | | | |
| Coco | — | — | — | 1 |
| monoethanolamide³ | | | | |
| Lauryl Hydroxysultaine⁴ | 4 | 4 | — | — |
| Sodium | — | — | 10 | 8 |
| Lauroamphoacetate⁵ | | | | |
| Para Hydroxy Phenyl | — | — | 3 | 3 |
| Butanone⁶ | | | | |
| Dipropylene Glycol | 10 | 7.5 | 5 | 5 |
| Guar, Hydroxylpropyl | 0.4 | 0.4 | 0.4 | 0.4 |
| Trimonium Chloride⁷ | | | | |
| Fragrance | 2.4 | 2.4 | 2.4 | 2.4 |
| Propellant A46⁸ | 4 | 4 | 4 | 4 |
| Preservatives, | Up to 1% | Up to 1% | Up to 1% | Up to 1% |
| pH adjusters | | | | |
| Sodium Chloride | Add as needed to adjust viscosity | | | |
| Water | Q.S. | Q.S. | Q.S. | Q.S. |

¹Sodium Laureth (1 molar ethylene oxide) sulfate
²Tegobetaine F-B, 30% active; supplied by Goldschmidt Chemical
³Coco monethanolamide at 85% active; supplied by Stephan Co or supplier Evonik
⁴LHS (Mackam LHS) at 42.5% active level; supplied by Rhodia
⁵NaLaa (Mackam HPL-ULS) at 22% active level; supplied by Rhodia
⁶Raspberry Ketone; supplied by Spectrum
⁷Jaguar C500, MW of 500,000, Charge Density of 0.8; supplied by Rhodia
⁸Aeron A-Blends, A46 (Isobutane/Propane = 84.85/15.15); supplied by Diversified CPC International

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A method of washing and conditioning hair comprising:
a. Applying and massaging a shampoo composition directly onto the scalp and root area of the hair, where root area is defined as 50% of the total length of the hair starting at the end attached to the scalp;
b. Applying a rinse-off conditioner composition on the length and ends of the hair, where length is defined as the hair starting below the root line at the back of the neck/base of the head and the ends are defined as the 4 cm of the hair furthest away from the scalp.
c. Rinsing the hair with water.

2. A method of washing and conditioning hair comprising:
a. Applying and massaging a shampoo composition directly onto the scalp and root area of the hair, where root area is defined as 50% of the total length of the hair starting at the end attached to the scalp;
b. Applying a second shampoo or cleansing composition to the length and ends, where length is defined as the hair starting below the root line at the back of the neck/base of the head and the ends are defined as the 4 cm of the hair furthest away from the scalp;
c. Rinsing the hair with water

3. A method of washing and conditioning hair comprising:
a. Applying and massaging a shampoo composition directly onto the scalp and root area of the hair, where root area is defined as 50% of the total length of the hair starting at the end attached to the scalp;
b. Rinsing the hair with water;
c. Applying a leave-on conditioner composition on the length and ends of the hair, where length is defined as the hair starting below the root line at the back of the neck/base of the head and the ends are defined as the 4 cm of the hair furthest away from the scalp.

4. A method according to claim 1 wherein the shampoo composition is followed by rinsing with water before the application of the rinse-off conditioner composition.

5. A method according to claim 2 where in the second shampoo or cleansing composition is a dilution of the first shampoo composition.

6. A method according to claim 1 wherein the shampoo composition is delivered via an applicator having a single and/or plurality of outlets extending more than 1 cm from its base.

7. A method according to claim 1 wherein the shampoo formulation composition has a zero shear viscosity value measured at 0.01 Pa of from less than about 20,000 Pa s.

8. A method according to claim 1 wherein the shampoo composition has a zero shear viscosity value measured at 0.01 Pa of from less than about 15,000 Pa s.

9. A method according to claim 1 wherein the shampoo composition has a zero shear viscosity value measured at 0.01 Pa of from about less than about 10,000 Pa s.

10. A method according to claim 1 wherein the shampoo composition is dispensed with a foam applicator.

11. A method according to claim 10 wherein the shampoo composition has a zero shear viscosity measures at 0.01 Pa s of less than about 15,000 Pa s.

12. A method according to claim 11 wherein the shampoo composition has a zero shear viscosity measures at 0.01 Pa s of less than about 10,000 Pa s.

13. A method according to claim 1 wherein the rinse-off conditioner is dispensed with a foam applicator.

14. A method according to claim 13 wherein the viscosity of the rinse-off conditioner composition has a zero shear viscosity value measured at 0.01 Pa of less than about 15,000 Pa s.

15. A method according to claim 13 wherein the viscosity of the rinse-off conditioner composition has a zero shear viscosity value measured at 0.01 Pa of less than about 10,000 Pa s.

16. A method according to claim 1 wherein the shampoo and rinse-off conditioner compositions are included in a single package having a dual chamber.

17. A method according to claim 1 wherein the shampoo composition is dispensed as a foam.

18. A method according to claim 17 wherein the shampoo composition is dispensed as aerosol foam.

19. A method according to claim 17 wherein the shampoo care composition is dispensed as a pumped foam.

20. A method according to claim 1 wherein the rinse-off composition is dispensed as a foam.

21. A method according to claim 1 wherein the rinse-off composition is dispensed as an aerosol foam.

22. A method according to claim 1 wherein the rinse-off composition is dispensed as a pumped foam.

23. A method according to claim 1 wherein the shampoo/conditioning composition comprises a benefit agent selected from the group consisting of an anti-dandruff agent, anti-fungal agent, anti-itch agent, anti-bacterial agent, anti-microbial agent, moisturization agent, anti-oxidant, vitamin, lipid soluble vitamin, chelant, perfume, brightener, enzyme, sensate, attractant, dyes, pigment, bleach, and mixtures thereof.

24. A method according to claim 23 wherein the shampoo composition comprises a sensate.

25. Instructions for complying with hair washing and conditioning method according to claim 1 comprising the steps of:
1. Wet hair thoroughly;
2. Apply shampoo directly onto scalp and root;
3. Continue application until entire surface area of the scalp and root are covered;
4. Massage into roots and scalp and avoid/minimize spreading it onto length and ends;
5. Rinse thoroughly;
6. Apply conditioner product to length and ends of the hair;
7. Rinse thoroughly.

26. A kit of products comprising one or more containers of a cleansing product, one or more containers of a conditioning product and usage instructions, wherein the cleansing product includes an attached or detached applicator with a single or plurality of outlets that extend more than 1 cm from its base and/or wherein the instructions suggest zone specific application.
